(19) United States
(12) Patent Application Publication
(10) Pub. No.: US 2017/0189674 A1
(43) Pub. Date: Jul. 6, 2017

(54) MEDICAL ELECTRICAL LEAD

(71) Applicant: Medtronic, Inc., Minneapolis, MN (US)

(72) Inventors: Toine Camps, Eys Wittem (NL); Jean Rutten, Gulpen (NL)

(21) Appl. No.: 15/365,301

(22) Filed: Nov. 30, 2016

Related U.S. Application Data
(60) Provisional application No. 62/274,493, filed on Jan. 4, 2016.

(51) Int. Cl. A61N 1/05; A61N 1/37

(52) U.S. Cl. CPC A61N 1/059; A61N 1/0563; A61N 1/371

(57) ABSTRACT

The present disclosure relates to an implantable medical device such as a medical electrical lead. The implantable medical device comprises an electrical connector assembly, an electrode, and an elongated lead body having a proximal end and a distal end. The lead body comprising an elongated conductor, a coiled conductor, and an insulative cover surrounding the coiled conductor. The insulative cover comprises a set of ports along a distal portion of the lead body and adjacent the electrode. The electrode is located on the lead body distal to the electrical connector assembly. The coiled conductor extends distally from the electrical connector assembly within the elongated lead body and is mechanically coupled to the electrode. The elongated conductor extends distally from the connector assembly and is electrically coupled to the electrode.

### CROSS-REFERENCE TO RELATED APPLICATIONS

This application claims the benefit of U.S. Provisional Application No. 62/274,493, filed on Jan. 4, 2016. The disclosure of the above application is incorporated herein by reference in its entirety.

### TECHNICAL FIELD

The present disclosure relates generally to implantable medical leads, and, more particularly, to epicardial medical electrical leads.

### BACKGROUND

The human anatomy includes many types of tissues that can either voluntarily or involuntarily, perform certain functions. After disease, injury, or natural defects, certain tissues may no longer operate within general anatomical norms. For example, after disease, injury, time, or combinations thereof, the heart muscle may begin to experience certain failures or deficiencies. Certain failures or deficiencies can be corrected or treated with implantable medical devices (IMDs), such as implantable pacemakers, implantable cardioverter defibrillator (ICD) devices, cardiac resynchronization therapy defibrillator devices, or combinations thereof.

IMDs detect and deliver therapy for a variety of medical conditions in patients. IMDs include implantable pulse generators (IPGs) or implantable cardioverter-defibrillators (ICDs) that deliver electrical stimuli to tissue of a patient. ICDs typically comprise, inter alia, a control module, a capacitor(s), and a battery that are housed in a hermetically sealed container with a lead extending therefrom. It is generally known that the hermetically sealed container can be implanted in a selected portion of the anatomical structure, such as in a chest or abdominal wall, and the lead can be inserted through various venous portions so that the tip portion can be positioned at the selected position near or in the muscle group. When therapy is required by a patient, the control module signals the battery to charge the capacitor, which in turn discharges electrical stimuli to tissue of a patient via electrodes disposed on the lead, e.g., typically near the distal end of the lead. Typically, a medical electrical lead includes a flexible elongated body with one or more insulated elongated conductors. Each conductor electrically couples a sensing and/or a stimulation electrode of the lead to the control module through a connector module.

In order to deliver stimulation or to perform sensing functions, it is desirable for the distal end of the lead to substantially remain in its position, as originally implanted by a physician. Typically, an endocardial lead is placed within the heart to deliver therapy; however, endocardial leads cannot be used for all types of patients. For example, some patients have inadequate vascular access for an endocardial lead and, therefore, may benefit from placement of an epicardial lead. Numerous epicardial leads have been designed. Exemplary epicardial leads include U.S. Pat. No. 6,010,526 B2, U.S. Pat. No. 7,270,669 B1, U.S. Pat. No. 8,150,535 and US2006466271A. It is desirable to develop additional epicardial lead designs.

### BRIEF DESCRIPTION OF THE DRAWINGS

The following drawings are illustrative of particular embodiments of the present invention and therefore do not limit the scope of the invention. The drawings are not to scale (unless so stated) and are intended for use in conjunction with the explanations in the following detailed description. Embodiments of the present invention will hereinafter be described in conjunction with the appended drawings, wherein like numerals denote like elements.

FIG. 1 is a conceptual schematic view of an implantable medical device in which a medical electrical lead extends therefrom.

FIG. 2 is a functional block diagram of the IMD shown in FIG. 1.

FIG. 3 is a perspective view of a medical electrical epicardial lead shown in FIG. 1.

FIG. 4 is a perspective cross-sectional view along a longitudinal axis of the epicardial lead shown in FIG. 3.

FIG. 5 is a perspective view of a distal end of a lead body.

FIG. 6 is a cross-sectional view of an exemplary lead body.

FIG. 7 is a perspective view of the epicardial medical electrical lead shown in FIG. 4.

FIG. 8A is a perspective cross-sectional view along a longitudinal axis of the medical electrical lead depicted in FIG. 7 including the proximal sensing electrode and the helical tip.

FIG. 8B is a perspective view of a distal end of the medical electrical lead, depicted in FIGS. 7-8, in which an enlarged distal helical electrode is shown.

FIG. 9 is a perspective view of a distal end of a conventional steerable guide catheter.

FIG. 10A depicts a pattern of holes in an exemplary insulative cover for the epicardial lead shown in FIGS. 7-8.

FIG. 10B depicts a cross-sectional view of an opposing pair of ports disposed in the insulative cover depicted in FIG. 10A.

FIG. 11 depicts a perspective view of the insulative cover shown in FIGS. 10A-10B of an exemplary lead body in which the insulative cover is laid flat along a xy axis for illustrative purposes since the cover is typically in a cylindrical form.

FIG. 12 depicts a perspective view of the epicardial medical electrical lead shown in FIG. 4.

### SUMMARY

The present disclosure is directed toward an implantable medical device that includes an electrical connector assembly, a sensing electrode, a pacing electrode, and an elongated lead body having a proximal end and a distal end. The lead body comprises an elongated conductor (also referred to as a cable), a coiled conductor, and an insulative cover surrounding the coiled conductor. The coiled conductor includes an inner lumen, which the elongated conductor is located. The insulated cover comprises a set of ports, located along a distal portion of the lead body, that expose a greater surface area of the sensing electrode to the body. The sensing electrode is located distal from the electrical connector assembly but proximal from the pacing electrode.

In one or more embodiments, the coiled conductor extends distally from the electrical connector assembly within the elongated lead body and is mechanically and not electrically coupled to the pacing electrode. The cable extends distally from the connector assembly and is electrically and mechanically coupled to the pacing electrode. The outer conductor coil extends distally from the connector assembly and is electrically and mechanically coupled to the sensing electrode.

In one or more embodiments, an implantable medical device includes an epicardial lead that comprises an elongated lead body defining a proximal end and a distal end. The lead body comprises an elongated conductor, a coiled conductor, a sensing electrode, and an insulative cover surrounding the coiled conductor. The insulative cover comprises a set of ports along an axial length at a distal portion of the lead body. The set of ports are adjacent to the sensing electrode.

Compared to conventional leads, the epicardial lead of the present disclosure may be more flexible and provide increased sensing capabilities due, at least in part, to a set of ports, formed in the insulated cover, and adjacent to a sensing electrode. The set of ports also assists to directly transfer torque to the tip.

### DETAILED DESCRIPTION

Epicardial leads can be beneficial to patients (e.g. pediatric patients, etc.) with limited vascular access. Epicardial leads have unrestricted access to optimal sites on the left ventricle or other cardiac tissue sites for delivery of electrical stimulation. The ability to place an epicardial lead in an optimal location may enhance delivery of therapies. Exemplary cardiac therapies that may employ the epicardial lead disclosed herein comprises cardiac resynchronization therapy (CRT), bradycardia pacing, or any other suitable pacing therapies.

The following description is merely exemplary in nature and is not intended to limit the present disclosure, application, or uses. The devices described herein include an exemplary number of leads, etc. One will understand that the components, including number and kind, may be varied without altering the scope of the disclosure. Also, devices according to various embodiments may be used in any appropriate diagnostic or treatment procedure, including a cardiac procedure. The epicardial leads disclosed herein are typically chronically implanted in a patient.

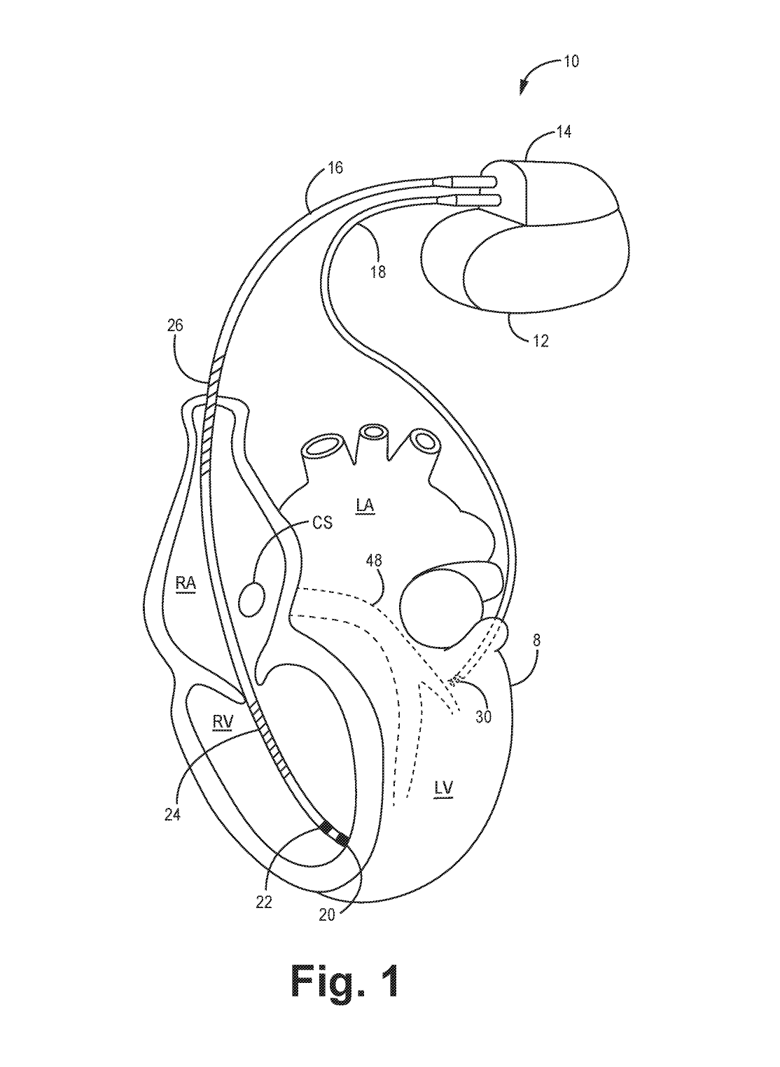

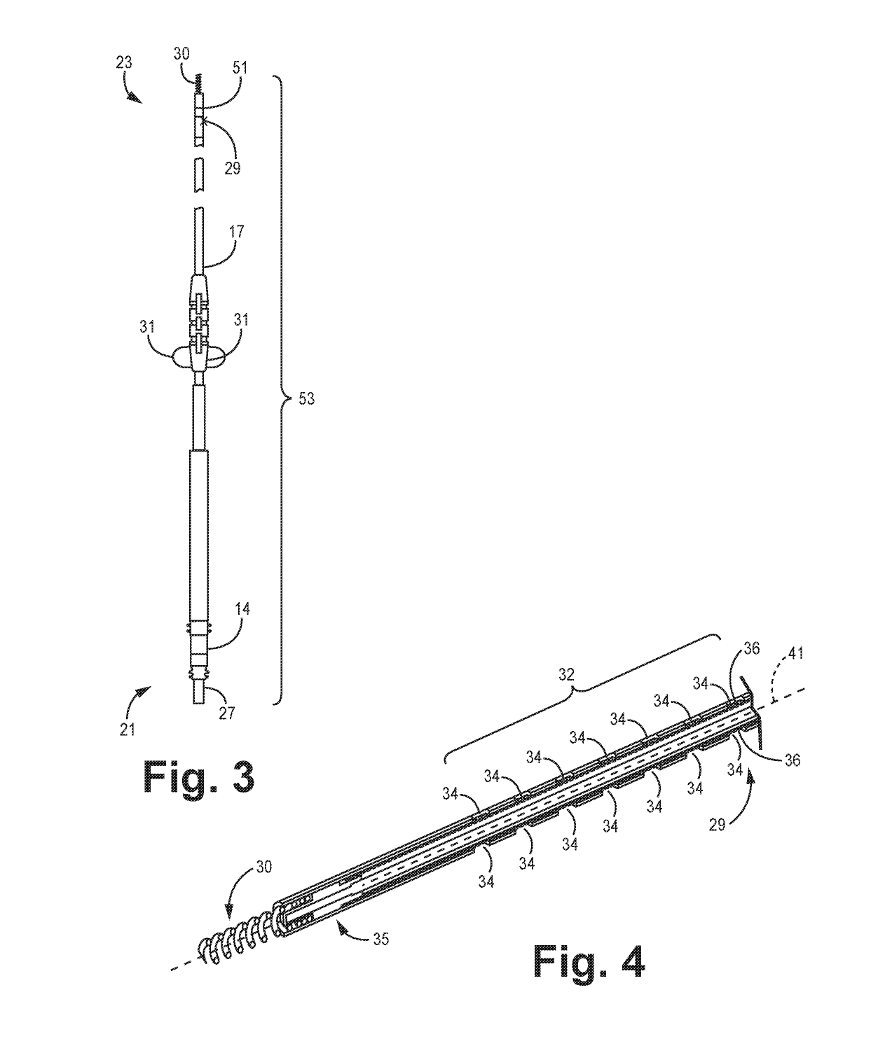

FIG. 1 depicts a medical device system 10 (also referred to as an implantable medical device (IMD)) coupled to a patient's heart 8 by way of a right ventricular (RV) lead 16 and an epicardial lead 18, each of which are stabilized through an anchoring sleeve 31, shown in FIG. 3. The anchoring sleeve 31 is used in a conventional fashion to stabilize the lead and seal the venous insertion site. A medical device system 10 includes a medical device housing 12 having a connector module 14 (e.g. international standard (IS)-1, defibrillation (DF)-1, IS-4 etc.) that electrically couples various internal electrical components housed in medical device housing 12 to a proximal end of a medical electrical lead 18. A medical device system 10 may comprise any of a wide variety of medical devices that include one or more medical lead(s) 18 (e.g. bipolar fixed screw lead) and circuitry coupled thereto. An exemplary medical device system 10 can take the form of an implantable cardiac pacemaker, an implantable cardioverter, an implantable defibrillator, an implantable cardiac pacemaker-cardioverter-defibrillator (PCD), a neurostimulator, a tissue and/or muscle stimulator. IMDs are implanted in a patient in an appropriate location. Exemplary IMDs are commercially available as including one generally known to those skilled in the art, such as the Medtronic CONCERTO™, SENSIA™, VIRTUOSO™, RESTORE™, RESTORE ULTRA™, VIVA™ sold by Medtronic, Inc. of Minnesota. Aspects of the disclosure can be used with many types and brands of IMDs. Medical device system 10 may deliver, for example, pacing, cardioversion or defibrillation pulses to a patient via electrodes disposed on distal end of one or more lead(s). Specifically, the lead may position one or more electrodes with respect to various cardiac locations so that medical device system 10 can deliver electrical stimuli to the appropriate locations. Lead 16 is a dual or single coil defibrillation lead that is attached to the endocardium, the innermost layer of tissue that lines the chambers of the heart. The endocardium underlies the much more voluminous myocardium. Lead 16 includes a RV elongated electrodes 24, 26 can be configured to sense electrical activity of a patient's heart during the delivery of pacing therapy. In the illustrated example, bipolar or unipolar electrodes 20, 22 (also referred to as RV electrodes) are located proximate to a distal end of the lead 16. The IMD 10 may deliver defibrillation shocks to the heart 8 via any combination of the elongated electrode 24, 26 and a housing electrode 12.

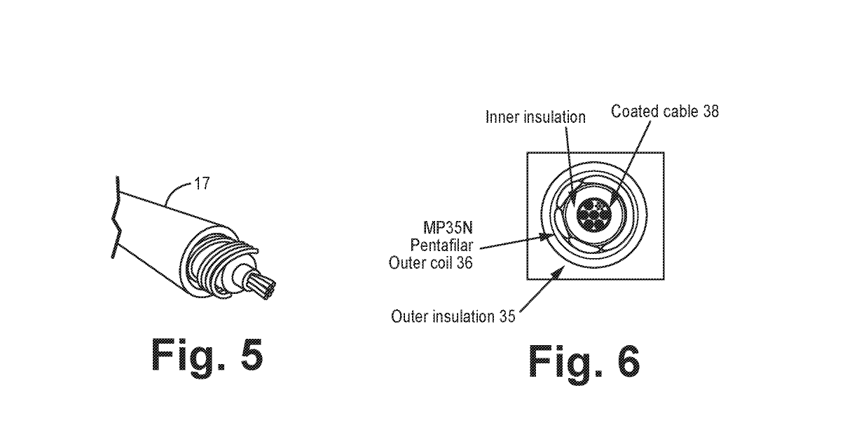

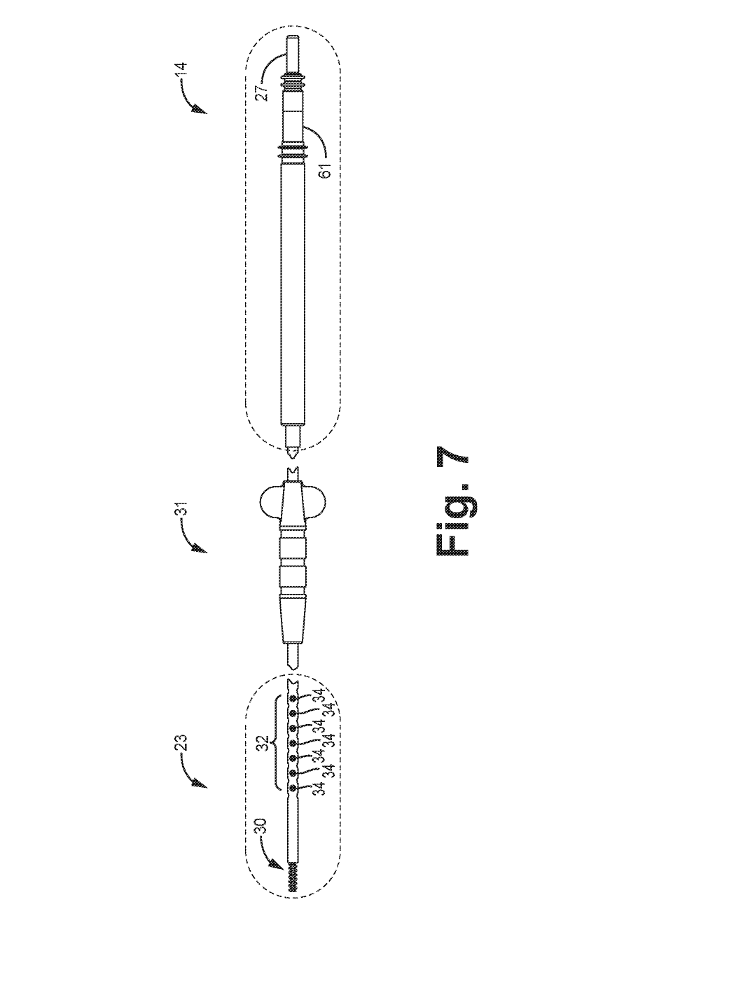

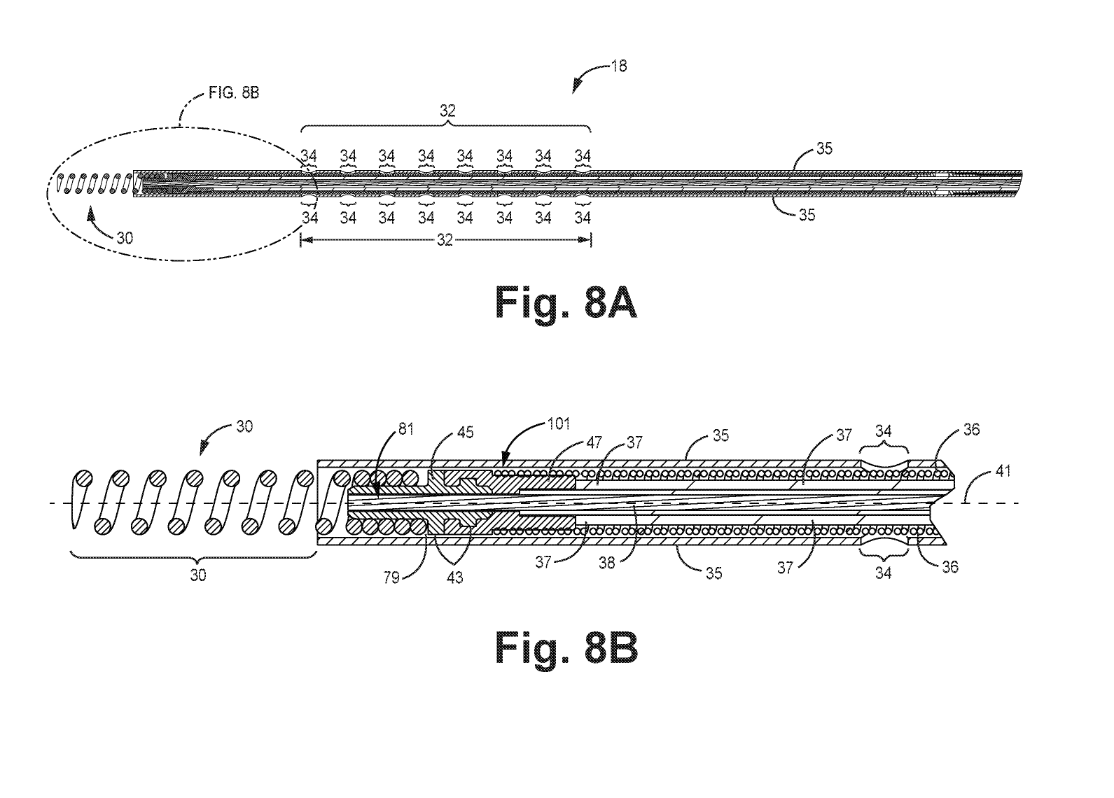

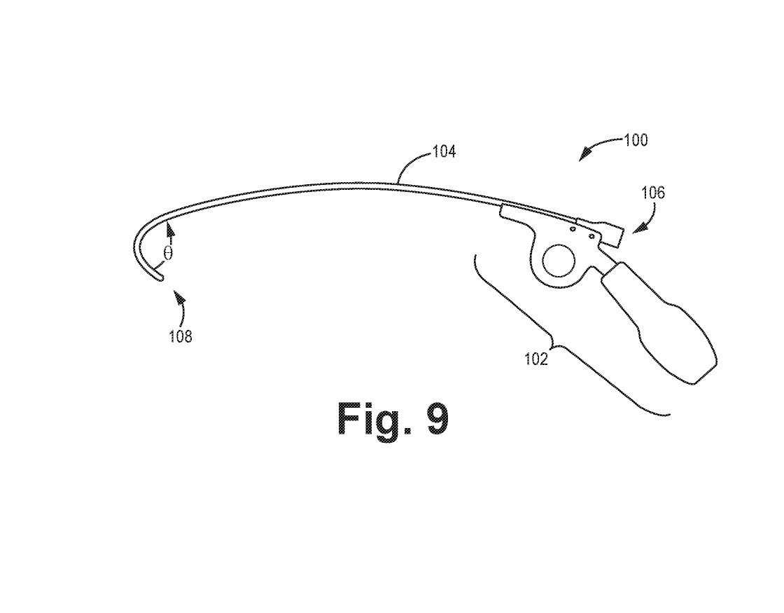

FIGS. 3-8, and 12 depict an exemplary medical electrical lead 18 of the present disclosure. Medical electrical lead 18 can be used as an epicardial lead that is delivered to target tissue through use of a guide catheter 100, as shown in FIG. 9. Catheter 100 is designed as a fixed shape to wrap around the surface of the heart in order to reach atria (left atrium (LA), right atrium (RA)) or ventricle (LV, RV). The LV veins 48 are shown in ghost lines to indicate that the LV veins are behind heart 8.

Lead 18 is loaded into inner lumen 106 of the guide catheter 100 while the user holds handle 102 (also referred to as a hub) and passes the lead through distal end 108. Pericardial access is attained through a supxiphoidal puncture with a small needle (e.g. Tuohy needle ranging in size from about 22G to about 25G). The guiding catheter 100 can be introduced into the pericardial space and the lead placed using any suitable means.

Lead 18 can be configured to deliver electrical stimulation to tissue and/or sense signals from the tissue in response to the delivery of electrical stimulation. An exemplary means in which to used the lead to deliver electrical stimulation is shown and described in Medtronic Inc. SELECTSURE Manual (2013) incorporated by reference in its entirety herein. Referring to FIGS. 3-4, lead 18 includes a distal end 23 and a proximal end 21 with a lead body 17 therebetween that generally defines a major longitudinal axis 41. The proximal end 21 of the lead 18 is connected to an in-line bipolar connector module 14 shown in FIG. 1. Briefly, the bipolar connector assembly 14, located on the proximal end 21 of the lead 18, carries two electrical connectors, a ring 61 and a pin 27 shown in FIG. 7. Referring to FIG. 8B, the elongated conductor 38 (also referred to as a cabled conductor or cable) is electrically connected to the pin 27 and to the pacing electrode 30 (also referred to as the helix, helical electrode, tip) at the distal end 23 of the lead body 17. Pacing electrode 30 can be configured as a cathode that operates in conjunction with an anode (i.e. ring electrode 29) to form a pacing vector. Exemplary tip to ring length can be 9 millimeters. Pin 27 is further connected to the electrical connector assembly 14.

The outer conductor 36, also referred to as a coiled conductor, provides mechanical strength for the lead body 17. The outer conductor 36 is electrically coupled to the connector ring 61 and is only mechanically coupled to the helix electrode 30 at the distal end 23 of the lead body 17. Outer conductor 36 is further mechanically connected to sleeve 47. The outer surface of the outer conductor 36 can be configured to serve as an anode. However, in alternate embodiments, the outer surface can be formed as a cathode.

The outer conductor 36 and the elongated conductor 38 (i.e. cable) have insulative layers 35 and 37, respectively, that can comprise one or more polymers. In one embodiment, the cable 38 is insulated with PTFE and silicone while polyurethane (PU) is used as insulation 35 for the coiled conductor 36. For example, polyurethane can be used and/or SI polyimide. The present disclosure can employ other polymers such as those which are shown and described with respect to U.S. Pat. No. 8,005,549 issued Aug. 23, 2011, U.S. Pat. No. 7,783,365 issued Aug. 24, 2010, and assigned to the assignee of the present invention, the disclosure of which are incorporated by reference in their entirety herein. Another exemplary insulative material that can be used is shown relative to SELECTSURE™ Model 3830 quadripolar lead, commercially available from Medtronic, Inc. located in Minnesota.

Insulation 35 includes set of ports 32 (FIG. 7) that expose the outer conductor 36 to the patient's body. Ports 34 do not substantially weaken the insulative cover 35 along the distal end of the lead 18. Ports 34 assist in creation of increased flexibility to bend or move the lead 18 into position compared to a conventional lead that lacks a set of ports.

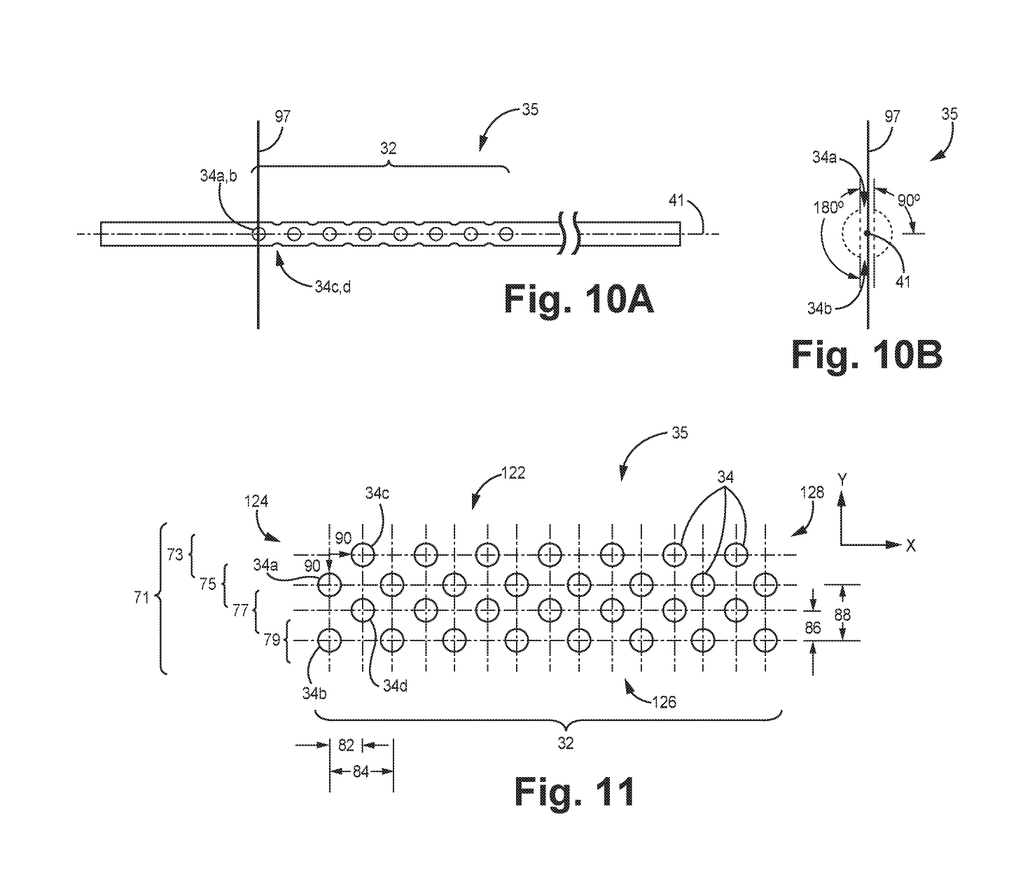

Details of the insulative cover 35 are shown in FIGS. 10-11. Referring to FIG. 11, insulative cover 35 is laid flat along a XY axes in order to better show the location of each port 34 relative to another port 34. Insulative cover 35 is configured to include a set of ports 34 (also referred to as apertures or holes) along the distal end 23 (FIG. 7) of lead 18 to allow one or more sensing electrodes to be exposed to the patient's body to sense physiological effects and/or electrical response to delivery, for example, of electrical stimulation from tip electrode 30. Set of ports 32, extends length 32 and a circumference 71, over the insulation 35 (e.g. tubing 55D of polyurethane).

The insulative cover 35 includes a first end 122, a second end 124, a third end 126, and a fourth end 128. A set of ports 32 is provided that comprise a first, second, third and fourth set of ports 73, 75, 77, 79, respectively placed along the X-axis. The first set of ports 73 are symmetrically spaced apart relative to the third set of ports 77, and the second set of ports 75 are symmetrically spaced apart relative to the fourth set of ports 79 along the Y-axis. Each port 34 in one plane is offset (e.g. up to 90 degrees away) from ports 34 in another plane.

Once the insulative cover 35 is wrapped or formed into a tube, sets of ports correspond with other sets of ports 34 that are diametrically opposed to the first sets of ports. For example, one set of ports 73 (also referred to as a first set of ports) align with the other set of ports 77 (also referred to as a second set of ports), as shown in FIG. 11. A third set of ports 73 align with the fourth set of ports 77.

Exemplary alignment of a pair of opposing ports 34ab is shown in FIGS. 10A-B and FIG. 11. One port 34a is circumferentially spaced apart by 180° to another port 34b, as shown in FIG. 10B. The centers of each pair of opposing ports 34ab lie within a plane 97 orthogonal to the axis 41. Each pair of opposing ports 34ab lies along an axis orthogonal to axis 41. The axis along which at least one pair of ports 34ab lies is offset 90 degrees from the axis along which at least one adjacent pair of ports 34cd lies.

After the insulation 35 is formed into a cylinder, one set of ports, shown in ghost lines in FIG. 10A, are orthogonal (e.g. 90 degrees offset) from another set of ports (shown as divets as exemplified by ports 34cd). Each port 34 is spaced apart by a pre-specified distance. Length 82 is the distance along the axial length between the ports which is 0.0039 inches (1 mm). Axial length is the length along the lead body 17. Length 84 is distance between the ports 34 in the same orientation along the X-axis and is double of the length 82. Length 86 is the distance of about 0.021 inches between the center of one port 34 to the center of another port 34 along adjacent sets of port 34. Length 88 is the distance between the center of one port 34 and another port 34 along the Y-axis and may be about 0.42 inches. The exemplary diameter of each port is 0.075 inches.

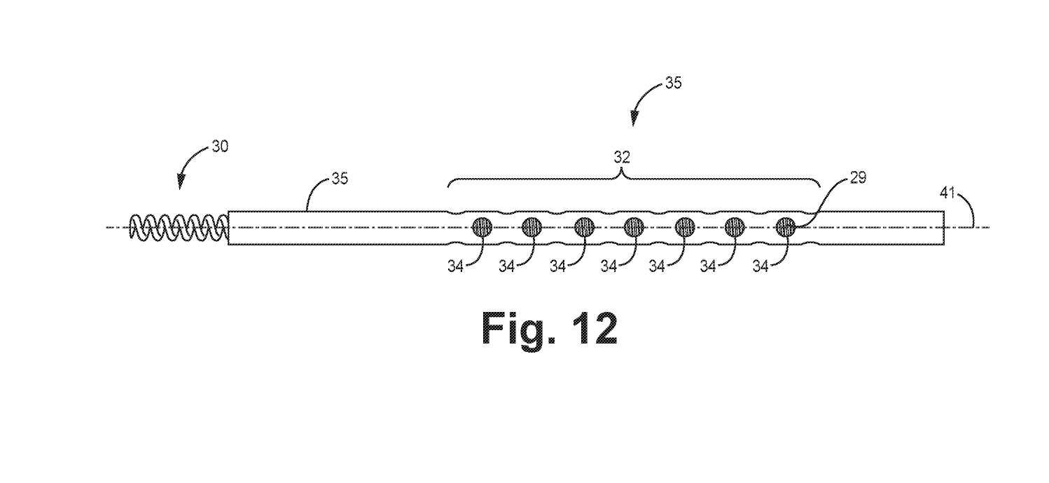

Referring to FIG. 12, each port 34 is positioned over a flexible electrode 29 (e.g. coil, anode ring etc.) that comprises platinum iridium coated with titanium nitride (TiN) for improved sensing performance. Flexible electrode 29 is mechanically and electrically coupled to the coiled conductor.

In one or more embodiments, each port is substantially circular in shape. In one or more other embodiments, each port is substantially non-circular in shape. In one or more embodiments, the set of ports are located such that a first set of ports are asymmetrically placed from a second set of ports.

Ports 34 can be formed using a mold or using a sharp puncturing tool to puncture a set of ports in the insulation. Each port 34 can be the same size. In one or more other embodiments, each port can be a different size from other ports. The electrode 29, solely used for sensing, is disposed along the longitudinal axis 41, adjacent the set of ports 70.

An outer conductor 36 (also referred to as a “conductor coil) extends the length of the lead body 17, running from the electrical connector module 14 at the proximal end of the lead 18 to an electrode 29 at or near the distal end 23 of the lead 18, as shown in FIG. 18B. In addition, the lead 18 is provided with a stranded conductor 38, preferably taking the form of a cable or a bundled stranded wire, which extends from the connector 14 to which the coil's conductor 36 is coupled distally to a point along the lead body 17, located distally. The distal end of the stranded conductor is mechanically, but not electrically, coupled to the coiled conductor 36, rendering the helical tip 30 to be solely used for active fixation with tissue. Since the outer conductor 36 is mechanically connected to the tip 30 but not electrically connected to tip 30, torque can be directly and/or completely transferred to the helical tip 30. Torque, applied through the electrical connector 14, is directly transferred to the tip 30 (i.e. helix) where the torque is actually needed in order to efficiently and/or effectively advance the helical tip 30 in the appropriate manner to allow the helical tip 30 to be securely screwed into epicardial myocardial tissue. In contrast, conventional epicardial leads include insulative covers, without ports, that causes any applied torque to be totally or substantially transferred by the tubing to the tip (i.e. helix).

Additionally, any type of flexible anodal ring, for sensing in one embodiment, causes the torque to become consumed by the coil (i.e. electrode sub-assembly) at that point and not directly transferred to the tip. The flexible anodal ring can comprise a platinum iridium coil. Optionally, one or more of the electrodes on the lead 18 can be drug eluting such as that which is disclosed in US 20140005762 filed Jun. 29, 2012, assigned to the assignee of the present invention, incorporated by reference in its entirety. Additionally, the tip and/or ring electrodes can be coated with titanium nitride (TiN). Electrodes are coated with TiN for improved pacing performance.

Optionally, a flexible anode ring electrode can be included on the lead. The flexible anode ring electrode can comprise bare Pt/Ir. The electrodes can take the form of ring and barrel shaped electrodes, respectively, as described in U.S. Pat. No. 8,825,180 by Bauer, et al., incorporated herein by reference in its entirety. The electrodes can include steroid (e.g. beclomethasone) eluting MCRD's. Other known electrode designs may of course be substituted.

Active fixation mechanism 30 (e.g. helix, tines, screw) is located at the distal end of lead 16 and/or 18 which attaches or screws into tissue. An exemplary helical tip 30 has a helical pitch with an outer diameter of 1.0 millimeters (mm) and a length of about 4 mm to screw into tissue. The helical tip 30 is positioned adjacent the target tissue and is fixated into the tissue by, for example, turning the proximal end a number of times (e.g. 5 times) while holding the connector 14 to transfer torque up to the helical tip 30. Once fixated, the user pushes and/or pulls on the lead 18 to confirm that lead 18 is fixated and not moving.

At the distal end of the lead 18, an integrated sleeve 43 supports and electrically separates the outer conductor 36 and the elongated conductor 38 (i.e. cable). The sleeve 43 comprises first and second components 45, 47, respectively. The first component 45 comprises platinum iridium and is directly connected to the elongated conductor 38. The second component 47 comprises a polymer such as polyurethane (e.g. 55 Durometer) that is overmolded onto the first component 45. By overmolding the polyurethane over the first component 45, the sleeve 43 becomes a single integrated component. The second component 47 is directly connected to the elongated conductor 38 and to the coiled conductor 36 thereby insulating the elongated conductor 38 from the coiled conductor 36. The helix 30 is welded onto sleeve 43 at site 79.

The flexible electrode 29 directly attaches to the sleeve. Flexible electrode 29 comes up on the outside of the polyurethane and butts up against ledge 101 of the lip. The cable goes through that sleeve that is spot welded to the cable on the distal end of that sleeve.

The outer conductor 36 is not electrically connected to the cable 38. The cable 38 extends through to the helical tip 30. The helix is the active component attached to the cable 38 on one electrical circuit referred to as a first circuit. The conductor coil 36, which wraps around the cable 38 that extends along the lead body 17, comes around and attaches to the flexible electrode 29 underneath the ports 34. The conductor end is welded to the sleeve 43 and sleeve 43 attaches to the flexible anode ring MP35N 29 underneath the portholes 34. Referring to FIG. 3, the tip to ring space 51 is shown between tip electrode 30 and flexible electrode 29 while the lead has an entire length of 53.

If the present invention is embodied in the form of an endocardial lead, then electrode assembly and electrode 29 may be replaced by corresponding structure from any conventional endocardial pacing or defibrillation lead, including those described in U.S. Pat. No. 5,456,705 issued to Morris, U.S. Pat. No. 5,282,844 issued to Stokes, U.S. Pat. No. 5,144,960 issued to Mehra, and U.S. Pat. No. 5,014,696 issued to Mehra, all incorporated by reference herein in their entireties.

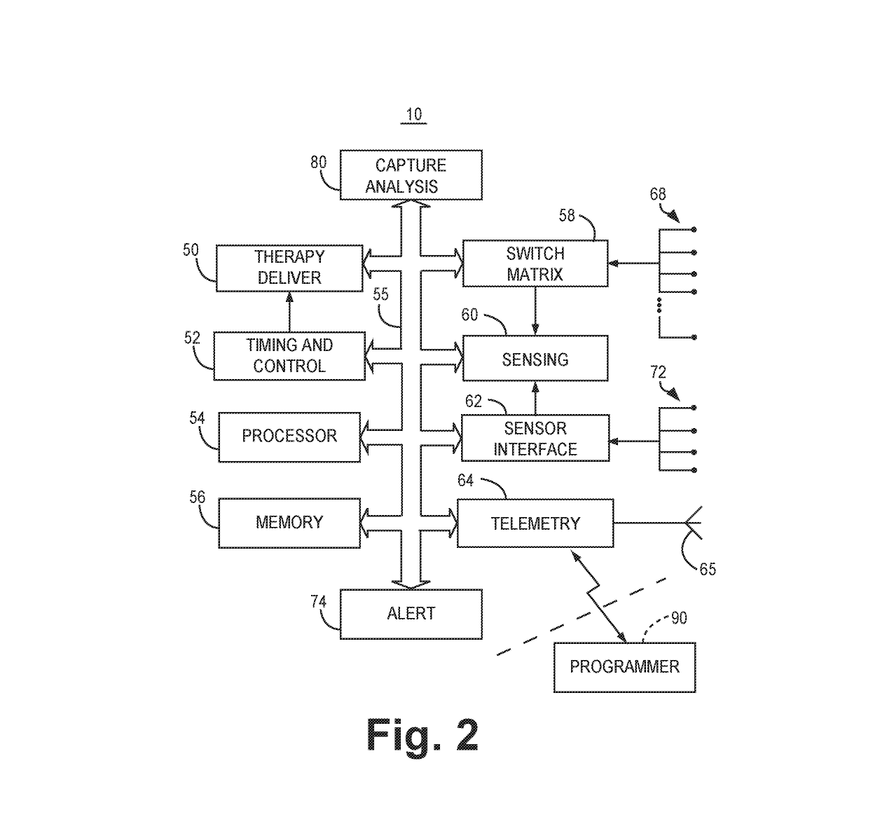

FIG. 2 is a functional block diagram of IMD 10. IMD 10 generally includes timing and control circuitry 52 and an operating system that may employ processor 54 for controlling sensing and therapy delivery functions in accordance with a programmed operating mode. Processor 54 and associated memory 56 are coupled to the various components of IMD 10 via a data/address bus 55. Processor 54, memory 56, timing and control 52, and capture analysis module 80 may operate cooperatively as a controller for executing and controlling various functions of IMD 10.

Processor 54 may include any one or more of a microprocessor, a controller, a digital state machine, a digital signal processor (DSP), an application specific integrated circuit (ASIC), a field-programmable gate array (FPGA), or equivalent discrete or integrated logic circuitry. In some examples, processor 54 may include multiple components, such as any combination of one or more microprocessors, one or more controllers, one or more DSPs, one or more ASICs, or one or more FPGAs, as well as other discrete or integrated logic circuitry. The functions attributed to processor 54 herein may be embodied as software, firmware, hardware or any combination thereof. In one example, capture analysis module 80 and/or sensing module 60 may, at least in part, be stored or encoded as instructions in memory 56 that are executed by processor 54.

IMD 10 includes therapy delivery module 50 for delivering a therapy in response to determining a need for therapy based on sensed physiological signals. Therapy delivery module 50 includes a signal generator for providing electrical stimulation therapies, such as cardiac pacing or arrhythmia therapies, including CRT. Therapies are delivered by module 50 under the control of timing and control 52. Therapy delivery module 50 is coupled to two or more electrodes 68 via a switch matrix 58 for delivering pacing pulses to the heart. Switch matrix 58 may be used for selecting which electrodes and corresponding polarities are used for delivering electrical stimulation pulses. Electrodes 68 may correspond to the electrodes 12, 20, 22, 24, 26, 30, shown in FIG. 1 or any electrodes coupled to IMD 10.

Timing and control 52, in cooperation with processor 54 and capture analysis module 80, control the delivery of pacing pulses by therapy delivery 50 according to a programmed therapy protocol, which includes the option of multi-site pacing wherein multiple pacing sites along a heart chamber are selected using methods described herein. Selection of multiple pacing sites and control of the pacing therapy delivered may be based on results of activation time measurements or an anodal capture analysis algorithm or a combination of both. As such, capture analysis module 80 is configured to determine pacing capture thresholds and detect the presence of anodal capture for determining both anodal and cathodal capture thresholds for a given pacing vector in some embodiments.

Electrodes 61 are also used for receiving cardiac electrical signals. Cardiac electrical signals may be monitored for use in diagnosing or monitoring a patient condition or may be used for determining when a therapy is needed and in controlling the timing and delivery of the therapy. When used for sensing, electrodes 68 are coupled to sensing module 60 via switch matrix 58. Sensing module 60 includes sense amplifiers and may include other signal conditioning circuitry and an analog-to-digital converter. Cardiac EGM signals (either analog sensed event signals or digitized signals or both) may then be used by processor 54 for detecting physiological events, such as detecting and discriminating cardiac arrhythmias, determining activation patterns of the patient's heart, measuring myocardial conduction time intervals, and in performing anodal capture analysis and pacing capture threshold measurements as will be further described herein.

IMD 10 may additionally be coupled to one or more physiological sensors 72. Physiological sensors 72 may include pressure sensors, accelerometers, flow sensors, blood chemistry sensors, activity sensors or other physiological sensors for use with implantable devices. Physiological sensors may be carried by leads extending from IMD 10 or incorporated in or on the IMD housing. Sensor interface 62 receives signals from sensors 72 and provides sensor signals to sensing module 60. In other embodiments, wireless sensors may be implanted remotely from IMD and communicate wirelessly with IMD 10. IMD 10 further includes IMD telemetry circuitry 64 and antenna 65. IMD telemetry circuitry 64 may receive sensed signals transmitted from wireless sensors. Sensor signals are used by processor 54 for detecting physiological events, conditions or triggering alert 74. Telemetry circuitry 64 and antenna 65 may correspond to telemetry systems known in the art. The operating system includes associated memory 56 for storing a variety of programmed-in operating mode and parameter values that are used by processor 54. The memory 56 may also be used for storing data compiled from sensed signals and/or relating to device operating history for telemetry out upon receipt of a retrieval or interrogation instruction. The processor 54 in cooperation with therapy delivery module 50, sensing module 60 and memory 56 executes an algorithm for measuring activation times for selecting pacing sites for delivering multi-site pacing.

A capture analysis algorithm may be stored in memory 56 and executed by processor 54 and/or capture analysis module 80 with input received from electrodes 68 for detecting anodal capture and for measuring pacing capture thresholds. Microprocessor 54 may respond to capture analysis data by altering electrode selection for delivering a cardiac pacing therapy. Data relating to capture analysis may be stored in memory 56 for retrieval and review by a clinician and that information may be used in programming a pacing therapy in IMD 10.

IMD 10 further includes telemetry circuitry 64 and antenna 65. Programming commands or data are transmitted during uplink or downlink telemetry between IMD telemetry circuitry 64 and external telemetry circuitry included in programmer 90. Alert 74 can be generated when IMD 10 when a preset threshold has been crossed.

Programmer 90 may be a handheld device or a microprocessor based home monitor or bedside programming device used by a clinician, nurse, technician or other user. IMD 10 and programmer 90 communicate via wireless communication. Examples of communication techniques may include low frequency or radiofrequency (RF) telemetry using Bluetooth or MICS but other techniques may also be used.

A user, such as a physician, technician, or other clinician, may interact with programmer 90 to communicate with IMD 10. For example, the user may interact with programmer 90 to retrieve physiological or diagnostic information from IMD 10. Programmer 90 may receive data from IMD 10 for use in electrode selection for CRT, particularly data regarding cathodal and anodal capture thresholds and other measurements used in electrode selection such as hemodynamic measurements and LV activation times. A user may also interact with programmer 90 to program IMD 10, e.g., select values for operational parameters of the IMD. For example, a user interacting with programmer 90 may select programmable parameters controlling a cardiac rhythm management therapy delivered to the patient's heart 8 via any of electrodes 68.

Processor 54, or a processor included in programmer 90, is configured to compute battery expenditure estimates in some embodiments. Using measured pacing capture thresholds and lead impedance measurements, along with other measured or estimated parameters, the predicted battery longevity of the IMD 10 may be computed for different pacing configurations. This information may be used in selecting or recommending a multi-site pacing configuration. As such, IMD 10 is configured to perform lead impedance measurements and determine other parameters required for estimated energy expenditure calculations, which may include but are not limited to a history of pacing frequency, capture thresholds, lead impedances, and remaining battery life.

While not shown explicitly in FIG. 2, a user may interact with programmer 90 remotely via a communications network by sending and receiving interrogation and programming commands via the communications network. Programmer 90 may be coupled to a communications network to enable a clinician using a computer to access data received by programmer 90 from IMD 10 and to transfer programming instructions to IMD 10 via programmer 90. Reference is made to commonly-assigned U.S. Pat. No. 6,599,250 (Webb et al.), U.S. Pat. No. 6,442,433 (Linberg et al.) U.S. Pat. No. 6,622,045 (Snell et al.), U.S. Pat. No. 6,418,346 (Nelson et al.), and U.S. Pat. No. 6,480,745 (Nelson et al.) for general descriptions and examples of network communication systems for use with implantable medical devices for remote patient monitoring and device programming, hereby incorporated herein by reference in their entirety.

The epicardial lead shown and described herein can be attached to any viable location on the heart. Exemplary locations include the LV, the right atrium, a backside of the heart, LV lateral wall and other suitable locations. Additionally, the lead body can be less than 7 French such as a 4 French or 4.1 French lead body.

While one or more embodiments have been generally described, other modifications can be made to make a lead that can find other useful applications. Exemplary embodiments are listed below.

Embodiment 1 is an implantable medical device comprising:

an electrical connector assembly;

a pacing electrode; and

an elongated lead body having a proximal end and a distal end, the lead body comprising an elongated conductor, a coiled conductor, and an insulative cover surrounding the coiled conductor, the insulative cover comprising a set of ports along a distal portion of the lead body and adjacent a sensing electrode; wherein

the pacing electrode is located on the lead body distal to the electrical connector assembly;

the coiled conductor extends distally from the electrical connector assembly within the elongated lead body and is mechanically coupled to the pacing electrode; and

the elongated conductor extends distally from the connector assembly and is electrically coupled to the pacing electrode.

Embodiment 2 is the implantable medical device of embodiment 1 wherein a port of the set of ports is substantially a same size as another port in the set of ports.

Embodiment 3 is the implantable medical device of embodiments 1 or 2 further comprising:

a sleeve coupled to the elongated conductor; and

a helical tip connected to a distal end of the sleeve.

Embodiment 4 is the implantable medical device of embodiments 1 through 3 wherein the sleeve comprises a first and second component, the first component comprising platinum iridium and the second component being a polymer, the second component directly connected to the elongated conductor and to the coiled conductor.

Embodiment 5 is the implantable medical device of embodiments 1 through 4 wherein the first component of the sleeve being directly connected to the elongated conductor.

Embodiment 6 is the implantable medical device of embodiments 1 through 5 wherein the sensing electrode is solely used for sensing.

Embodiment 7 is the implantable medical device of embodiments 1 through 6 wherein the set of ports in the lead body expose the coiled conductor to a patient's body.

Embodiment 8 is the implantable medical device of embodiments 1 through 7 wherein the torque is directly transferred to the tip through the coiled conductor.

Embodiment 9 is the implantable medical device of embodiments 1 through 8 wherein the insulative cover with the set of ports transfers a portion of torque to the tip.

Embodiment 10 is the implantable medical device of embodiments 1 through 9 wherein the set of ports comprises a first set of ports and a second set of ports offset from the first set of ports.

Embodiment 11 is the implantable medical device of embodiments 1 through 10 The implantable medical device of claim 8 wherein offset is defined as the first set of ports being 90 degrees away from the second set of ports.

Embodiment 12 is the implantable medical device of embodiments 1 through 11 wherein each port is substantially circular in shape.

Embodiment 13 is the implantable medical device of embodiments 1 through 12 wherein each port is substantially non-circular in shape.

Embodiment 14 is the implantable medical device of embodiments 1 through 13 wherein the set of ports are located such that a first set of ports are symmetrically placed from a second set of ports.

Embodiment 15 is the implantable medical device of embodiments 1 through 14 wherein the set of ports are located such that a first set of ports are asymmetrically placed from a second set of ports.

Embodiment 16 is the implantable medical device of embodiments 1 through 15 wherein the coiled conductor is mechanically and not electrically coupled to the pacing electrode.

Embodiment 17 is the implantable medical device of embodiments 1 through 16 wherein the elongated conductor is mechanically and electrically coupled to the pacing electrode.

Embodiment 18 is the implantable medical device of embodiments 1 through 17 wherein the coiled conductor is mechanically and electrically coupled to the sensing electrode.

Embodiment 19 is the implantable medical device of embodiments 1 through 18 wherein the coiled conductor is not electrically connected to the pacing electrode.

Although the present invention has been described in considerable detail with reference to certain disclosed embodiments, the disclosed embodiments are presented for purposes of illustration and not limitation and other embodiments of the invention are possible. It will be appreciated that various changes, adaptations, and modifications may be made without departing from the spirit of the invention and the scope of the appended claims.

Embodiment 20 is an implantable medical device comprising:

an implantable medical electrical lead comprising:
an elongated lead body defining a proximal end and a distal end, the lead body comprising a coiled conductor, a sensing electrode coupled to the coiled conductor, and an insulative cover surrounding the coiled conductor, the insulative cover comprising a set of ports along an axial length of a distal portion of the lead body, the set of ports being adjacent to the sensing electrode.

Embodiment 21 is a device as in embodiment 20, wherein the lead performs sensing.

What is claimed is:

1. An implantable medical device comprising:
an implantable medical electrical epicardial lead comprising:
an elongated lead body defining a proximal end and a distal end, the lead body comprising a coiled conductor, a sensing electrode coupled to the coiled conductor, and an insulative cover surrounding the coiled conductor, the insulative cover comprising a set of ports along an axial length of a distal portion of the lead body, the set of ports being adjacent to the sensing electrode to allow the sensing electrode to be exposed.

2. The device of claim 1 further comprising:
a pacing electrode coupled to the lead body;
the coiled conductor extending distally from the proximal end and mechanically coupled to the pacing electrode; and
an elongated conductor extending distally from the proximal end and electrically coupled to the pacing electrode.

3. The device of claim 1 further comprising:
a plurality of pairs of opposing ports are spaced along the distal portion of the lead body.

4. The device of claim 3 further comprising:
centers of each pair of ports lay within a plane orthogonal to a longitudinal axis defined by the lead body.

5. The device of claim 3 wherein each pair of ports lies along an axis orthogonal to the longitudinal axis.

6. The device of claim 1 wherein an axis along which at least one pair of ports lies is offset 90 degrees from the axis along which at least one adjacent pair of ports lies.

7. The implantable medical device of claim 1 wherein a port of the set of ports is substantially a same size as another port in the set of ports.

8. The implantable medical device of claim 1 wherein a port of the set of ports is a different size as another port in the set of ports.

9. The implantable medical device of claim 1 further comprising:
a sleeve defining a proximal end and a distal end, the sleeve coupled to the elongated conductor; and
a helical tip connected to the distal end of the sleeve.

10. The implantable medical device of claim 9 wherein the sleeve comprises a first and second component, the first component comprising platinum iridium and the second component being a polymer, the second component directly connected to the elongated conductor and to the coiled conductor.

11. The implantable medical device of claim 10 wherein the first component of the sleeve being directly connected to the elongated conductor.

12. The implantable medical device of claim 1 wherein the sensing electrode is solely used for sensing.

13. The implantable medical device of claim 1 wherein the set of ports in the insulative cover exposes the coiled conductor to a patient's body.

14. The implantable medical device of claim 11 wherein torque is directly transferred from the coiled conductor to the tip.

15. The implantable medical device of claim 14 wherein the insulative cover with the set of ports transfers a portion of torque to the tip.

16. The implantable medical device of claim 1 wherein the set of ports comprises a first set of ports and a second set of ports offset from the first set of ports.

17. The implantable medical device of claim 14 wherein offset is defined as the first set of ports being 90 degrees away from the second set of ports.

18. The implantable medical device of claim 1 wherein each port is substantially circular in shape.

19. The implantable medical device of claim 1 wherein each port is substantially non-circular in shape.

20. The implantable medical device of claim 1 wherein the set of ports are located such that a first set of ports are symmetrically placed from a second set of ports.

21. The implantable medical device of claim 1 wherein the set of ports are located such that a first set of ports are asymmetrically placed from a second set of ports.

22. The implantable medical device of claim 1 wherein the coiled conductor is mechanically and not electrically coupled to the pacing electrode.

23. The implantable medical device of claim 1 wherein the elongated conductor is mechanically and electrically coupled to the pacing electrode.

24. The implantable medical device of claim 1 wherein the coiled conductor is mechanically and electrically coupled to the sensing electrode.

25. The implantable medical device of claim 1 wherein the coiled conductor is not electrically connected to the pacing electrode.

26. An implantable medical lead comprising:
an electrical connector assembly;
a pacing electrode;
a sensing electrode; and
an elongated lead body having a proximal end and a distal end, the lead body comprising an elongated conductor, a coiled conductor, and an insulative cover surrounding the coiled conductor, the insulative cover comprising a set of ports along a distal portion of the lead body and adjacent the sensing electrode to allow the sensing electrode to be exposed, the set of ports comprising a first set of ports, a second set of ports, a third set of ports and a fourth set of ports, the first set of ports are symmetrically placed relative to the second set of ports, and the third set of ports are symmetrically placed relative to the fourth set of ports; the pacing electrode is located on the lead body distal to the electrical connector assembly;
the coiled conductor extends distally from the electrical connector assembly within the elongated lead body and is mechanically coupled to the pacing electrode and electrically connected to the sensing electrode; and
the elongated conductor extends distally from the connector assembly and is electrically coupled to the pacing electrode.

27. A method of using an implantable medical device comprising:
delivering electrical stimulation to tissue through a medical electrical lead comprising an elongated lead body, the lead body defining a proximal end and a distal end, the lead body comprising a coiled conductor, a sensing electrode, and an insulative cover surrounding the coiled conductor, the insulative cover comprising a set of ports along a distal portion of the lead body and adjacent the sensing electrode to allow the sensing electrode to be exposed; and
using the sensing electrode that is exposed to a patient's body through the set of ports to sense a response to the delivered electrical stimulation.

28. The method of claim 27 wherein electrical stimulation is delivered through
a pacing electrode coupled to the lead body;
the coiled conductor extending distally from the proximal end and mechanically coupled to the pacing electrode; and
an elongated conductor extending distally from the proximal end and electrically coupled to the pacing electrode.

29. The method of claim 27 wherein a plurality of pairs of opposing ports are spaced along the distal portion of the lead body.

30. The method of claim 29 wherein centers of each pair of ports lay within a plane orthogonal to a longitudinal axis defined by the lead body.

31. The method of claim 29 wherein each pair of ports lies along an axis orthogonal to the longitudinal axis.

32. The method of claim 29 wherein an axis along which at least one pair of ports lies is offset 90 degrees from the axis along which at least one adjacent pair of ports lies.

33. The method of claim 29 wherein a port of the set of ports is substantially a same size as another port in the set of ports.

34. The method of claim 27 wherein a port of the set of ports is a different size as another port in the set of ports.
